(19) United States
(12) Patent Application Publication
(10) Pub. No.: US 2015/0196209 A1
(43) Pub. Date: Jul. 16, 2015

(54) CARDIOVASCULAR RISK FACTOR SENSING DEVICE

(71) Applicant: Microsoft Corporation, Redmond, WA (US)

(72) Inventors: Daniel Scott Morris, Bellevue, WA (US); Desney S. Tan, Kirkland, WA (US); Timothy Scott Saponas, Woodinville, WA (US); Paul Henry Dietz, Redmond, WA (US); Andrew David Wilson, Seattle, WA (US); Alice Jane Bernheim Brush, Bellevue, WA (US); Erin Rebecca Griffiths, Charlottesville, VA (US)

(21) Appl. No.: 14/522,915

(22) Filed: Oct. 24, 2014

Related U.S. Application Data
(60) Provisional application No. 61/927,796, filed on Jan. 15, 2014.

(51) Int. Cl. A61B 5/021; A61B 5/0452; A61B 5/024; A47C 15/00

(52) U.S. Cl. CPC A61B 5/02141; A47C 15/004; A61B 5/0452; A61B 5/02416

(57) ABSTRACT

Various technologies described herein pertain to sensing cardiovascular risk factors of a user. A chair includes one or more sensors configured to output signals indicative of conditions at site(s) on a body of a user. A seat of the chair, a back of the chair, and/or arms of the chair can include the sensor(s). Moreover, the chair includes a collection circuit configured to receive the signals from the sensor(s). A risk factor evaluation component is configured to detect a pulse wave velocity of the user based on the signals from the sensor(s). The risk factor evaluation component is further configured to perform a pulse wave analysis of the user based on a morphology of a pulse pressure waveform of the user, and the pulse pressure waveform is detected based on the signals from the sensor(s).

### CROSS-REFERENCE TO RELATED APPLICATION

This application claims priority to U.S. Provisional Patent Application No. 61/927,796, filed on Jan. 15, 2014, and entitled “CHAIR THAT SENSES CARDIOVASCULAR RISK FACTORS”, the entirety of which is incorporated herein by reference.

### BACKGROUND

Heart disease is a leading cause of death in the United States, accounting for around 600,000 deaths in 2011 alone (e.g., nearly 31% of reported deaths in the United States). High blood pressure (hypertension, arterial hypertension), which is a chronic medical condition where blood pressure in arteries is elevated, is a well understood risk factor for heart disease. Hypertension can place persistent strain on the heart, and can lead to hypertensive heart disease, coronary artery disease, etc. if untreated. However, hypertension rarely has visible warning signs or symptoms; thus, people oftentimes fail to realize that they have hypertension.

Lack of awareness of a person experiencing hypertension is unfortunate because hypertension is typically treatable via lifestyle changes and/or medication. Lifestyle changes, such as changes in diet and exercise, can be effective in preventing the progression of hypertension by controlling blood pressure, which can decrease the risk of health complications. Moreover, numerous medications are available to treat hypertension. Therefore, a barrier to reducing the number of heart disease related deaths may be awareness of the risk (e.g., awareness of the person having hypertension).

Despite unawareness of hypertension being common, blood pressure readings have not gained much attention in the consumer space, presumably due to the intrusiveness of the measurement devices. For instance, inflatable cuffs of sphygmomanometers may be bulky, uncomfortable, and interfering to activities of a person, which can lead to lack of incorporation of such metering devices into products in the consumer space. Instead, hypertension typically continues to be identified for some people through infrequent screening (e.g., at an annual exam, health fair, etc.) or when seeking healthcare for an unrelated medical issue, while such cardiovascular risk for other people commonly is unassessed.

### SUMMARY

Described herein are various technologies that pertain to sensing cardiovascular risk factors of a user. A chair can include one or more sensors configured to output signals indicative of conditions at one or more sites on a body of a user. A seat of the chair, a back of the chair, and/or arms of the chair can include the one or more sensors. Moreover, the chair can include a collection circuit configured to receive the signals from the one or more sensors. A risk factor evaluation component can be configured to detect a pulse wave velocity of the user based on the signals from the one or more sensors. The risk factor evaluation component can further be configured to perform a pulse wave analysis of the user based on a morphology of a pulse pressure waveform of the user. Moreover, the pulse pressure waveform can be detected based on the signals from the one or more sensors.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

FIG. 1 illustrates a functional block diagram of an exemplary system that senses cardiovascular risk factors of a user.

FIG. 2 illustrates a functional block diagram of another exemplary system that senses cardiovascular risk factors of a user.

FIG. 3 illustrates a side view of an exemplary chair that senses cardiovascular risk factors in accordance with various embodiments.

FIG. 4 illustrates a front view of the exemplary chair of FIG. 3 that senses cardiovascular risk factors.

FIG. 5 illustrates a side view of another exemplary chair that senses cardiovascular risk factors.

FIG. 6 illustrates a side view of the exemplary chair of FIG. 5 in a reclined position.

FIG. 7 illustrates a top view of an exemplary arm of a chair that senses cardiovascular risk factors.

FIG. 8 is a flow diagram that illustrates an exemplary methodology for sensing cardiovascular risk factors.

FIG. 9 illustrates an exemplary computing device.

FIG. 10 illustrates an exemplary computing system.

### DETAILED DESCRIPTION

Various technologies pertaining to sensing cardiovascular risk factors using sensors of a chair are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details. In other instances, well-known structures and devices are shown in block diagram form in order to facilitate describing one or more aspects. Further, it is to be understood that functionality that is described as being carried out by certain system components may be performed by multiple components. Similarly, for instance, a component may be configured to perform functionality that is described as being carried out by multiple components.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

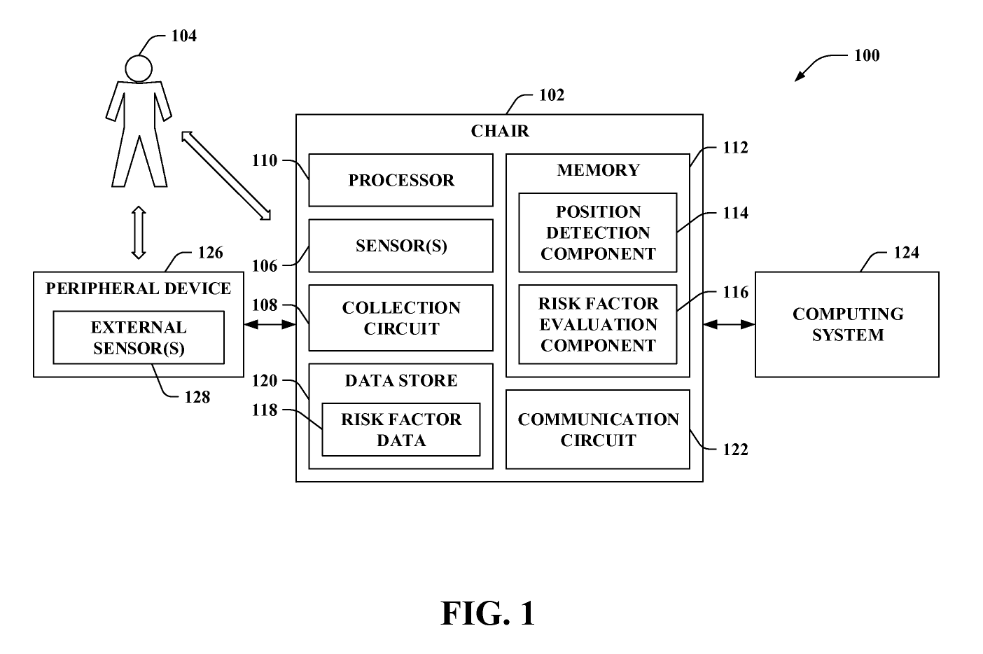

Referring now to the drawings, FIG. 1 illustrates a system 100 that senses cardiovascular risk factors of a user 104. The system 100 includes a chair 102 that senses aspects of cardiovascular wellness (e.g., cardiovascular risk factors) of the user 104. The aspects of cardiovascular wellness of the user 104 sensed by the chair 102 can correlate with hypertension and cardiovascular risk.

The chair 102 can provide mechanisms for assessing cardiovascular health of the user 104, where such mechanisms place little burden on the user 104, yet may have a beneficial impact on preventative monitoring of the cardiovascular risk factors of the user 104. By monitoring such risk factors, the user 104 can be encouraged to visit a clinic or make lifestyle changes if deemed to be at risk based on risk factor data 118 generated by the chair 102.

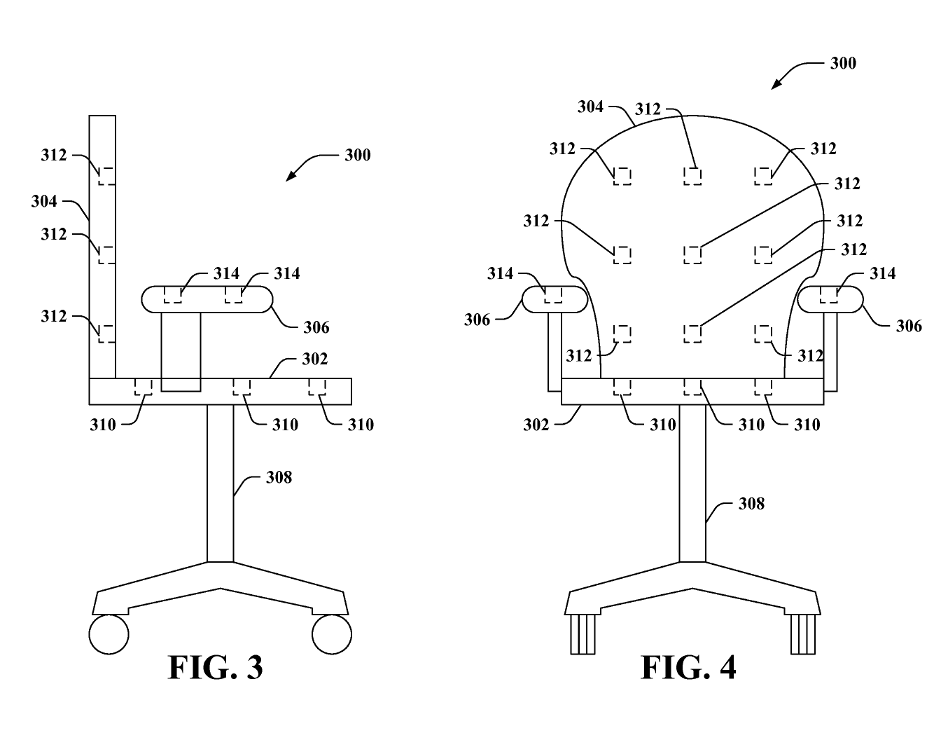

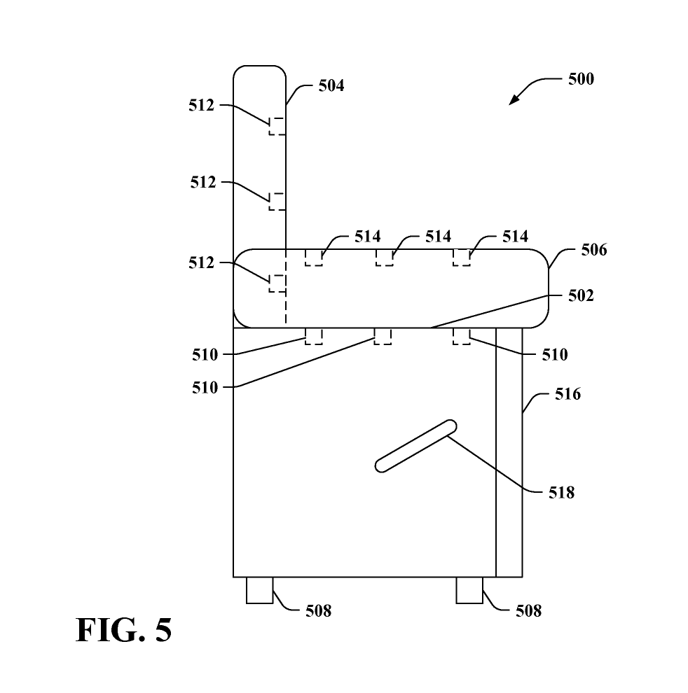

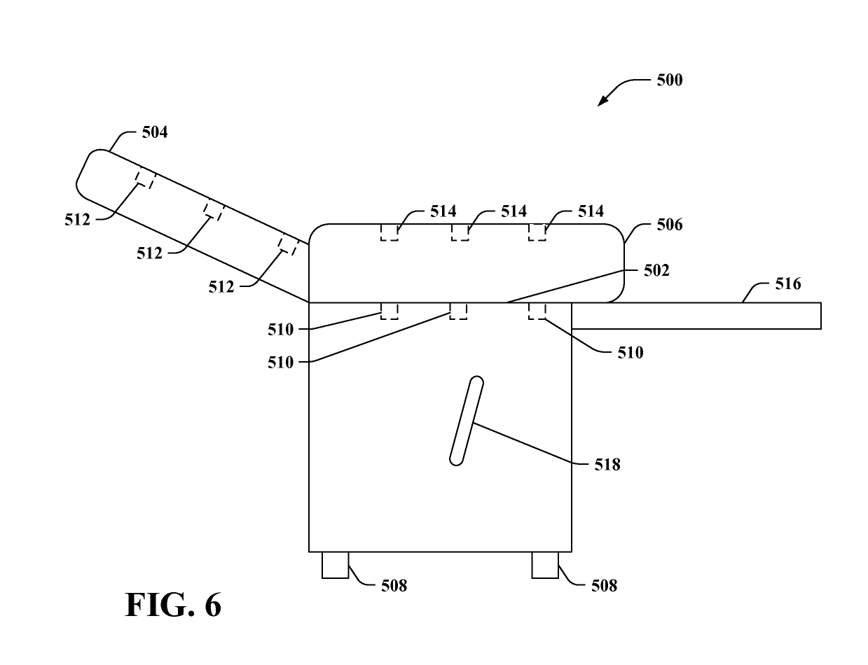

The chair 102 includes a seat (e.g., a seat 302 of FIGS. 3-4, a seat 502 of FIGS. 5-6, etc.), which can be supported by substantially any type of support (e.g., plurality of legs, support structure with caster wheels, etc.). Optionally, the chair 102 can include a back (e.g., a back 304 of FIGS. 3-4, a back 504 of FIGS. 5-6, etc.). The chair 102 also can optionally include arms (e.g., arms 306 of FIGS. 3-4, arms 506 of FIGS. 5-6, etc.).

Moreover, it is to be appreciated that substantially any type of chair 102 is intended to fall within the scope of the hereto appended claims. The chair 102, for instance, can be a stool, an armchair, a recliner, a seat, (e.g., a permanently fixed chair in a train, theater, airplane, etc.), a bicycle saddle, a car seat, a wheelchair, a toilet, or the like. Moreover, the chair 102 can allow for more than one person to sit thereupon; examples of such types of chairs include a couch, a sofa, a bench and so forth. Further, it is to be appreciated that many of the examples set forth herein can be extended to a bed instead of the chair 102 (e.g., the bed can similarly monitor the cardiovascular risk factors); however, the claimed subject matter is not so limited.

The chair 102 includes one or more sensor(s) 106 configured to output signals indicative of conditions at one or more site(s) on a body of the user 104. It is contemplated that the chair 102 can include substantially any number of sensor(s) 106. Each of the sensor(s) 106 can output a signal indicative of a condition at a respective site on the body of the user 104. The one or more sites on the body of the user 104 can include site(s) on a torso of the user 104, site(s) on lower limbs of the user 104, site(s) on upper limbs of the user 104, site(s) on a neck of the user 104, etc. The chair 102 can provide a ubiquitous, consumer friendly opportunity to sense conditions from the body of the user 104. According to an example, the signals from the sensor(s) 106 may be combined with various wearable and/or environmental sensors to provide a view of the cardiovascular health of the user 104; however, it is to be appreciated that the claimed subject matter is not limited to such example.

The chair 102 can include one or more types of sensor(s) 106. Examples of the types of the sensor(s) 106 include pressure sensors, optical sensors, (e.g., cameras), electrical sensors, acoustic sensors, Doppler ultrasound sensors, and so forth. In accordance with various embodiments, the chair 102 can include a plurality of differing sensor types (e.g., the sensor(s) 106 can include pressure sensor(s) and optical sensor(s), etc.). Locations of the sensor(s) 106 within the chair 102 and/or combinations of types of sensor(s) 106 included in the chair 102 are described in greater detail herein.

The chair 102 further includes a collection circuit 108 configured to receive the signals from the sensor(s) 106. The collection circuit 108 can receive the signals from the sensor(s) 106 in substantially any manner. For instance, the signals generated by the sensor(s) 106 can be received by the collection circuit 108 via wired connection(s), wireless connection(s), a combination thereof, and so forth. Moreover, the collection circuit 108 can synchronize the signals from the sensor(s) 106 (e.g., align the signals from the sensor(s) 106 in time); yet, according to other examples, it is contemplated that the risk factor evaluation component 116 can additionally or alternatively synchronize the signals from the sensor(s) 106.

According to various examples, the chair 102 can include at least one processor 110 and memory 112. The processor 110 described herein can include one or more processors and/or one or more processor cores. The processor 110 is configured to execute instructions loaded into the memory 112 (e.g., one or more components loaded into the memory 112 are executable by the processor 110, etc.). As described in greater detail herein, the memory 112 can include a risk factor evaluation component 116; thus, the risk factor evaluation component 116 can be executable by the processor 110. Additionally or alternatively, the memory 112 can include a position detection component 114; accordingly, the position detection component 114 can be executable by the processor 110.

The chair 102 (e.g., the memory 112) can optionally include the position detection component 114. According to an example, the position detection component 114 can be configured to detect presence or absence of the user 104 sitting on the chair 102. Following this example, the signals from the sensor(s) 106 can be analyzed by the position detection component 114 to identify whether the chair 102 is empty or the user 104 is sitting in the chair 102. Further, the position detection component 114 can determine a time at which the user 104 sits on the chair 102 or stands up from the chair 102. Moreover, the position detection component 114 can be configured to recognize the user 104 sitting in the chair 102, where the user 104 is identified out of a set of possible users (e.g., detect whether a first user or a second user is sitting in the chair 102, recognize the user 104 based upon the data from the sensor(s) 106, etc.). For instance, the user 104 can be recognized based upon measured weight of the user 104, a signature from the risk factor data 118 detected from the user 104, biometrics of the user 104, and so forth.

Pursuant to another example, the position detection component 114 can be configured to determine a position of the user 104 relative to the chair 102. For instance, the position detection component 114 can detect the position of the user 104 relative to the chair 102 based upon the signals obtained from the sensor(s) 106. By way of illustration, a signal from a first sensor that is part of a seat of the chair 102 versus a signal from a second sensor that is part of the seat of the chair 102, where the first sensor and the second sensor are at different locations within the seat, can be evaluated by the position detection component 114 to identify the relative position of the user 104 with respect to the chair 102. Moreover, the position detection component 114 can be configured to identify sites on the body of the user 104 from the detected position (e.g., the position detection component 114 can determine that a particular sensor from the sensor(s) 106 is aligned with a femoral artery of the user 104 and can capture signals indicative of a condition at the femoral artery whereas the position detection component 114 can determine that a disparate sensor from the sensor(s) 106 is not aligned with the femoral artery of the user 104, etc.). The position detection component 114 can further be configured to measure a distance between sites on the body of the user 104 (e.g., based upon particular sensors from the set of sensor(s) 106 identified as being aligned with the sites on the body of the user 104).

In accordance with yet another example, the chair 102 can be an adjustable chair (e.g., a recliner, etc.); following this example, the position detection component 114 can be configured to detect a configuration of the chair 102 or change in the configuration of the chair 102. By way of illustration, the position detection component 114 can detect whether a recliner is in an upright position or a reclined position and/or when the recliner transitions between the upright and reclined positions (or vice versa).

The chair 102 (e.g., the memory 112) can further include the risk factor evaluation component 116 configured to analyze the signals obtained by the collection circuit 108 from the sensor(s) 106 to infer various metrics pertaining to cardiovascular risk factors of the user 104. Thus, the risk factor evaluation component 116 can generate the risk factor data 118 for the user 104 over time. Moreover, the risk factor data 118 for the user 104 can be retained in a data store 120 of the chair 102. Examples of cardiovascular risk factors (e.g., the risk factor data 118) that can be inferred by the risk factor evaluation component 116 based on the signals from the sensor(s) 106 include, for example, pulse wave velocity (e.g., pulse transit time), pulse pressure waveform (e.g., pulse profile, properties of the waveform such as augmentation index, etc.), thoracic impedance (e.g., properties such as cardiac output, systemic vascular resistance, aortic blood flow velocity, etc.), heart rate, heart rate variability, respiration, a combination thereof, and so forth.

According to various examples, the risk factor evaluation component 116 can be configured to detect a pulse wave velocity of the user 104 based on the signals from the sensor(s) 106. The pulse wave velocity can provide a measure of arterial stiffness. The pulse wave velocity is a rate at which a pressure wave moves through a vessel of the user 104. The risk factor evaluation component 116 can detect a duration of time for a pressure wave to travel between two sites on the body of the user 104, referred to as the pulse transit time. Further, the risk factor evaluation component 116 can compute the pulse wave velocity based on a distance between the two sites and the pulse transit time.

Pursuant to various examples, the risk factor evaluation component 116 can be configured to perform a pulse wave analysis of the user 104 based on a morphology of a pulse pressure waveform of the user 104. Further, the risk factor evaluation component 116 can detect the pulse pressure waveform based on the signals from the sensor(s) 106. The morphology of the pulse pressure waveform includes shape features of the pulse pressure waveform and locations (e.g., in time and pressure) of waveform landmarks. The shape features of the pulse pressure waveform include high-level shape features such as a derivative and a peak-to-peak amplitude of the pulse pressure waveform. The waveform landmarks include a systolic peak, a diastolic foot, a dicrotic notch, and an anacrotic notch. The risk factor evaluation component 116 can be configured to derive a pulse pressure, a blood pressure (with calibration), an augmentation index, and/or a systolic ejection time of the user 104 via the pulse wave analysis. In contrast to the chair 102 enabling the pulse wave analysis of the user 104 to be performed, conventional approaches oftentimes perform a pulse wave analysis using a pressure sensor placed in (e.g., using a catheter) or above (e.g., using an applanation tonometer) an artery.

According to other examples, the risk factor evaluation component 116 can be configured to detect the pulse wave velocity of the user 104 based on the signals from the sensor(s) 106 and perform the pulse wave analysis of the user 104 based on the signals from the sensor(s) 106. Moreover, the risk factor evaluation component 116 can also be configured to detect other cardiovascular risk factors based on the signals from the sensor(s) 106.

The chair 102 can further include a communication circuit 122 configured to communicate with a computing system 124 (e.g., via a wired connection, a wireless connection, etc.). Accordingly, the communication circuit 122 can send the risk factor data 118 to the computing system 124. The computing system 124 can display the risk factor data 118 on a display screen of the computing system 124, send the risk factor data 118 received from the chair 102 to substantially any other computing system (e.g., a computing system of a doctor of the user 104, a computing system of a manager of the user 104, a health monitoring service, etc.), alter operation of a machine or vehicle based upon the risk factor data 118, or the like. Pursuant to another example, the computing system 124 can be included in a health monitoring system. The health monitoring system can maintain medical records for the user 104; the health monitoring system can restrict access to the medical records (e.g., allow the user 104 to access the medical records, allow other user(s) to access the medical records of the user 104 as specified by the user 104, etc.).

The computing system 124, for example, can be or include a desktop computing device, a server computing device (or plurality of server computing devices), a mobile computing device, a computing device in a health monitoring system, a computing device included in a machine or vehicle (e.g., an onboard computing device of a car, plane, truck, motorcycle, crane, forklift, tractor, etc.), or the like. A mobile computing device, for instance, can be a mobile phone (e.g., smartphone), a wearable computing device, a tablet computer, a laptop computer, a handheld computer, a personal digital assistant (PDA), a portable gaming device, and so forth.

Moreover, the system 100 optionally includes a peripheral device 126 that includes external sensor(s) 128, where the peripheral device 126 can mechanically and/or communicatively connect to the chair 102. According to an example, the peripheral device 126 can be a physically connected peripheral device that mechanically connects to the chair 102. An illustration of a physically connected peripheral device is a handlebar of an exercise bike or bicycle. Additionally or alternatively, the peripheral device 126 can be a digitally connected peripheral device that communicatively connects with the chair 102. Examples of a digitally connected peripheral device include an instrumented keyboard, a wrist based device (e.g., a wearable wrist based device such as a watch, a bracelet, etc.) that includes optical sensor(s) and/or pressure sensor(s), or an external camera. The peripheral device 126 can send signals captured by the external sensor(s) 128 to the chair 102 (e.g., the collection circuit 108) via wired connection(s), wireless connection(s), a combination thereof, and so forth.

Similar to the sensor(s) 106, the external sensor(s) 128 of the peripheral device 126 can output signals indicative of conditions at one or more sites on the body of the user 104. Moreover, the peripheral device 126 can send such signals to the chair 102; thus, the collection circuit 108 can receive the signals from the external sensor(s) 128 (e.g., via the communication circuit 122). Accordingly, the risk factor evaluation component 116 of the chair 102 can evaluate the signals from a combination of the sensor(s) 106 of the chair 102 and the external sensor(s) 128 of the peripheral device 126 to generate the risk factor data 118 for the user 104. Likewise, the data received from the external sensor(s) 128 can be utilized by the position detection component 114.

While testing of blood pressure is a common way to evaluate cardiovascular risk, people oftentimes fail to have their blood pressure measured or infrequently have their blood pressure measured. In contrast, the chair 102 enables measuring cardiovascular risk factors in a convenient, continuous manner (e.g., the risk factor data 118 can be continuously generated by the risk factor evaluation component 116 while the user 104 sits on the chair 102). Thus, use of the chair 102 enables the cardiovascular risk factors to be measured more frequently, without use of a bulky, painful, and intrusive cuff of a sphygmomanometer commonly used for measuring blood pressure.

The chair 102 can measure various indicators of cardiovascular risk that are analogous to blood pressure. Further, the chair 102 enables sensing variations in the cardiovascular risk factors of the user 104 over time, trends in the risk factor data 118 of the user 104, etc. The changes over time can be detected by the chair 102 due to the user 104 sitting in the chair 102 for an extended period of time. Moreover, the chair 102 enables the risk factor data 118 to be measured for a user who may otherwise be unaware of his or her cardiovascular risk.

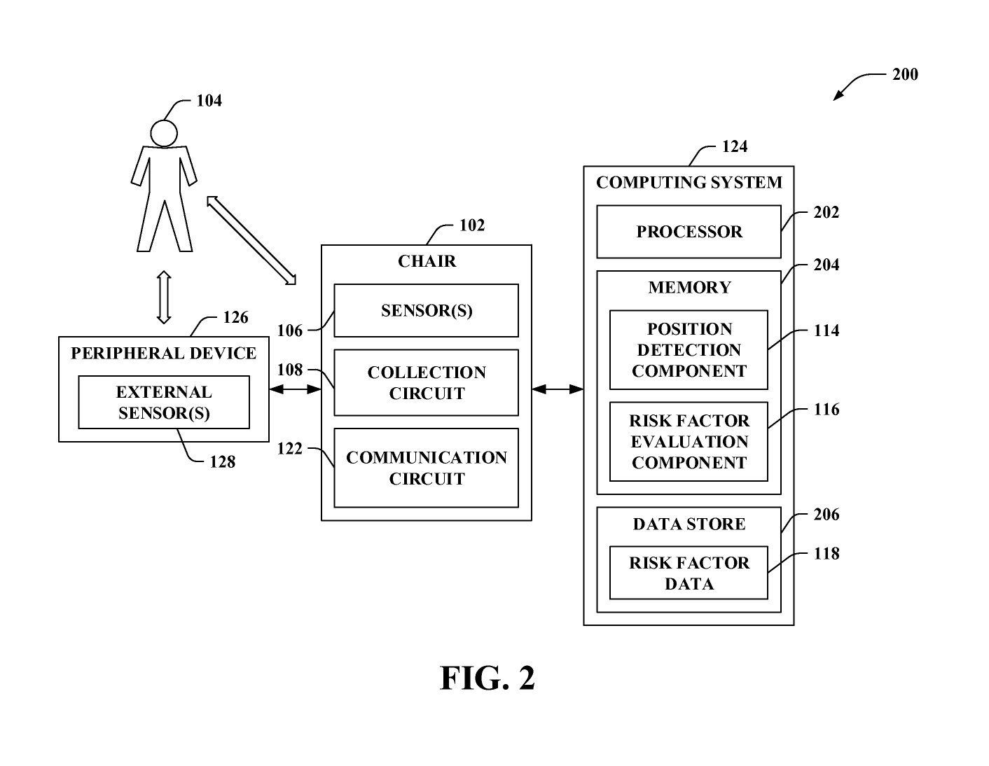

Turning to FIG. 2, illustrated is a system 200 that senses cardiovascular risk factors of the user 104. The system 200 includes the chair 102 and the computing system 124. Moreover, the system 200 optionally includes the peripheral device 126.

According to the example shown in FIG. 2, the chair 102 includes the sensor(s) 106, the collection circuit 108, and the communication circuit 122. The sensor(s) 106 can output signals indicative of conditions at one or more sites on the body of the user 104. Further, the collection circuit 108 can receive the signals from the sensor(s) 106 and/or the external sensor(s) 128 of the peripheral device 126. Moreover, the communication circuit 122 can send the signals from the sensor(s) 106 and/or the external sensor(s) 128 to the computing system 124.

As depicted, the computing system 124 can include at least one processor 202, memory 204, and a data store 206. The processor 202 can include one or more processors and/or one or more processor cores. The processor 202 is configured to execute instructions loaded into the memory 204 (e.g., one or more components loaded into the memory 204 are executable by the processor 202, etc.). According to the example shown in FIG. 2, the memory 204 includes the risk factor evaluation component 116; thus, the risk factor evaluation component 116 can be executable by the processor 202. Moreover, the memory 204 optionally includes the position detection component 114, and accordingly, the position detection component 114 can be executable by the processor 202. Again, the position detection component 114 can detect the position of the user 104 on the chair 102, etc. Further, the risk factor evaluation component 116 can generate the risk factor data 118; the risk factor data 118 generated by the risk factor evaluation component 116 can be retained in the data store 206 of the computing system 124.

Turning to FIGS. 3-4, illustrated is an exemplary chair 300 (e.g., the chair 102 of FIGS. 1-2) configured to sense cardiovascular risk factors in accordance with various embodiments. FIG. 3 depicts a side view of the chair 300, and FIG. 4 shows a front view of the chair 300. The exemplary chair 300 of FIGS. 3-4 includes a seat 302, a back 304, and arms 306. It is contemplated that the arms 306 are optional. Moreover, it is to be appreciated that the back 304 is optional. While the chair 300 is depicted as an office chair, it is to be appreciated that this example can be extended to substantially any other type of chair.

The seat 302 is further attached to a support 308. The seat 302 can be directly or indirectly attached to the support 308. The support 308 depicted in the illustrated example includes a number of caster wheels. Yet, it is to be appreciated that substantially any other type of support can be included as part of the chair 300 (e.g., the support 308 can include a plurality of legs, etc.).

The seat 302, the back 304, and the arms 306 of the chair 300 can be made of substantially any type of material. For instance, the seat 302, the back 304, and the arms 306 can be made of the same type of material, differing types of materials, a combination thereof, and so forth.

The seat 302 can be a center seat made of a solid material (e.g., the solid material forms a surface of the seat 302 on which a user sits) or an open center seat (e.g., soft material is attached to a frame where the soft material forms the surface of the seat 302 on which the user sits). A center seat (e.g., the seat 302), for example, can be made of materials such as solid wood, wood slats, padded leather (e.g., a solid base covered with padding and surrounded by leather), stuffed fabric (e.g., a solid base covered with padding and surrounded by fabric), metal, molded plastic, stone, or the like. Moreover, examples of materials for an open center seat (e.g., the seat 302) include woven wicker, leather, fabric, tape, canning, rush, reed, metal, woven wire, metal mesh, or the like.

Moreover, the chair 300 includes one or more sensors (e.g. the sensor(s) 106). The one or more sensors can be embedded in or attached to the seat 302, the back 304, and/or the arms 306. According to an example, at least a portion of external surface(s) of the seat 302, the back 304, and/or the arms 306, where the external surface(s) can contact a user who sits on the chair 300, can be at least a part of one or more of the sensors. For instance, a top surface of a sensor can be part of the external surface of the seat 302, the back 304, and/or the arms 306. According to another illustration, the external surface of the seat 302, the back 304, and/or the arms 306 can be formed of a conductive material, where the conductive material can be part of a sensor.

More particularly, the seat 302 of the chair 300 can optionally include one or more sensors 310. Further, the back 304 of the chair 300 can optionally include one or more sensors 312. Moreover, the arms 306 of the chair 300 can optionally include one or more sensors 314. While the sensors are depicted in the drawings as cubes, it is to be appreciated that any size and/or shape sensor is intended to fall within the scope of the hereto appended claims. Further, the chair 300 can optionally be mechanically and/or communicatively connected to a peripheral device that includes one or more external sensors (e.g., the peripheral device 126 including the external sensor(s) 128).

According to an example, the seat 302 can include the one or more sensors 310 (e.g., the one or more sensors 310 can be part of the seat 302). The one or more sensors 310 can be embedded in the seat 302 (e.g., between the solid base and fabric or leather of a center seat, etc.). For instance, a top surface of a sensor (e.g., one of the one or more sensors 310) can be exposed on the external surface of the seat 302. Pursuant to another illustration, a material from which the seat 302 is constructed can cover the one or more sensors 310 (e.g., a pressure sensor can be positioned below a layer of fabric or leather, an optical sensor can be positioned below a layer of transparent fabric, etc.). In accordance with yet another example, the one or more sensors 310 can be attached to the seat 302 (e.g., on top of the external surface of the seat, attached to a bottom surface of material used to form an open center seat, attached between wood slats, etc.). Moreover, the external surface of the seat 302 may be formed of material that is part of the one or more sensors 310. Similar to the foregoing description of the one or more sensors 310 being part of the seat 302, it is contemplated that the one or more sensors 312 can be part of the back 304 and/or the one or more sensors 314 can be part of the arms 306.

Moreover, it is contemplated that the seat 302, the back 304, and/or the arms 306 can include any number of sensors, which can be spaced relative to one another in the chair 300 in substantially any manner. For instance, as shown in FIG. 4, the back 304 of the chair 300 can include an array of nine sensors 312 spaced there across. It is to be appreciated, however, that the claimed subject matter is not limited to the depicted number and/or relative locations of the sensors 312 (e.g., the back 304 can lack sensors, the back 304 can include a single sensor, the sensors 312 need not be located in a three-by-three array, etc.).

Set forth below are various exemplary embodiments of the chair 300. Each of the exemplary embodiments described below includes a set of sensors that can be included in the chair 300. Moreover, it is to be appreciated that a combination of two or more of the sets of sensors described in the following exemplary embodiments can be included in the chair 300.

### Exemplary Embodiment 1

The chair 300 includes the sensors 310 that are part of the seat 302 of the chair 300. More particularly, the sensors 310 include one or more pressure sensors that are part of the seat 302. A weight of the user sitting on the seat 302 of the chair 300 can provide a consistent baseline pressure measured by the one or more pressure sensors. Moreover, the one or more pressure sensors can detect changes in the baseline pressure over time.

The one or more pressure sensors that are part of the seat 302 of the chair 300 can output signals indicative of conditions at site(s) on the body of the user. Further, the risk factor evaluation component 116 can be configured to detect a pulse pressure waveform of the user based on the signals from the one or more pressure sensors that are part of the seat 302. The pulse pressure waveform can be detected as part of a pulse wave analysis performed by the risk factor evaluation component 116. Accordingly, the risk factor evaluation component 116 can be configured to perform the pulse wave analysis of the user based on a morphology of the pulse pressure waveform.

For instance, the risk factor evaluation component 116 can infer a pulse profile based on the signals from the one or more pressure sensors that are part of the seat 302. The pulse profile can be a shape of a plot of pressure over time indicative of a pulse wave at the femoral artery of the user sitting on the chair 300. The one or more pressure sensors can output signals indicative of conditions at the femoral artery of the user sitting on the chair 300, thereby enabling detection of the pulse profile by the risk factor evaluation component 116.

Moreover, the risk factor evaluation component 116 can determine the pulse pressure, blood pressure, augmentation index, and/or systolic ejection time of the user based on a morphology of the pulse pressure waveform. For instance, an augmentation index can be derived from the morphology of the pulse pressure waveform (e.g., the augmentation index can be generated based on pressure changes in and/or near the femoral artery). The augmentation index is a property of the pressure wave that correlates with cardiac load, and consequently, risk of heart attack.

### Exemplary Embodiment 2

The chair 300 includes electrical sensors that are part of the arms 306 (e.g., the sensors 314 include electrical sensors) and/or the collection circuit 108 of the chair 300 is configured to receive signals from electrical sensors that are part of a peripheral device communicatively connected to the chair 300 (e.g., the peripheral device can be a physically connected peripheral device and/or a digitally connected peripheral device). In combination with the foregoing sensors, the chair 300 additionally includes pressure sensors that are part of the seat 302 (e.g., the sensors 310 include pressure sensors), optical sensors that are part of the seat 302 (e.g., the sensors 310 include optical sensors), and/or optical sensors that are part of the back 304 (e.g., the sensors 312 include optical sensors).

In accordance with this embodiment, the risk factor evaluation component 116 can be configured to detect a pulse wave velocity (e.g., pulse transit time, the risk factor data 118) of a user based on signals from the electrical sensors that are part of the arms and/or signals from the electrical sensors that are part of the peripheral device. Additionally, the risk factor evaluation component 116 can be configured to detect the pulse wave velocity of the user based on signals from the pressure sensors that are part of the seat 302, signals from the optical sensors that are part of the seat 302, and/or signals from the optical sensors that are part of the back 304. The pulse wave velocity (e.g., the pulse transit time) is a property of the pressure wave that can correlate with arterial stiffness, and thus cardiovascular risk.

To compute the pulse wave velocity, an electrocardiogram (EKG) signal of the user can be measured based on the signals from the electrical sensors (e.g., part of the arms 306 of the chair 300 and/or the peripheral device). The EKG signal of the user can be measured using electrical contact points on opposite sides of the heart (e.g., electrical sensors that are part of each of the arms 306 of the chair 300, electrical sensors on both ends of a handlebar of an exercise bike at which the user holds the handlebar, electrical sensors in a keyboard that the user can contact with his or her hand, wrist, or forearm while typing or otherwise using the keyboard, etc.). Based on the EKG signal, the risk factor evaluation component 116 detects a first time at which a pulse leaves a heart of the user. Moreover, the risk factor evaluation component 116 infers a second time at which the pulse arrives at a different site on the body of the user based on the signals from the pressure sensors and/or or optical sensors (e.g., part of the seat 302 and/or the back 304). The risk factor evaluation component 116 can compute a pulse transit time as a difference between the first time and the second time. Further, the risk factor evaluation component 116 can compute the pulse wave velocity based on the pulse transit time and a distance between the heart and the different site on the body of the user.

By way of illustration, the electrical sensors that are part of the arms 306 of the chair 300 can output signals used to detect when a pulse leaves the heart. Further, optical sensors and/or pressure sensors that are part of the seat 302 can output signals used to detect when the pulse reaches the femoral artery. The risk factor evaluation component 116 can compute the difference between such times to determine the pulse transit time.

### Exemplary Embodiment 3

The chair 300 includes optical sensors that are part of the back 304 (e.g., the sensors 312 include optical sensors, the optical sensors can be spread throughout the back 304). Optionally, the chair 300 can also include optical sensors that are part of the seat 302 and/or pressure sensors that are part of the seat 302 (e.g., the sensors 310 include optical sensors and/or pressure sensors).

In accordance with this embodiment (and similar to exemplary embodiment 2), the risk factor evaluation component 116 can be configured to detect the pulse wave velocity (e.g., the pulse transit time). According to exemplary embodiment 3, the risk factor evaluation component 116 can be configured to detect the pulse wave velocity of the user based on signals from the optical sensors that are part of the back 304. According to another example, the risk factor evaluation component 116 can be configured to detect the pulse wave velocity of the user based on the signals from the optical sensors that are part of the back 304 as well as signals from the optical sensors that are part of the seat 302 and/or signals from the pressure sensors that are part of the seat 302.

Similar to exemplary embodiment 2, the optical sensors and/or pressure sensors can be spread throughout the chair 300. Thus, the risk factor evaluation component 116 can infer when the pulse arrives at various sites on the body of the user. In accordance with exemplary embodiment 3, the risk factor evaluation component 116 can determine the pulse transit time without utilizing electrical sensors. Rather, two pressure sensors and/or optical sensors can indicate when the pulse passed two corresponding sites on the body of the user. Accordingly, the difference between the two times can be computed by the risk factor evaluation component 116 as the pulse transit time.

By way of illustration, a first optical sensor directed towards an upper part of user's back can detect when the pulse passes a first site, and a second optical sensor directed towards a lower part of the user's back can detect when the pulse passes a second location. The difference in the times at which the pulse is detected is determined to be the pulse transit time. An optical sensor, for example, can detect a time of arrival of a pulse through clothing of the user. Moreover, it is to be appreciated that a pressure sensor that is part of the seat 302 can similarly be employed to detect a time of arrival of the pulse at a corresponding site on the body of the user.

### Exemplary Embodiment 4

The chair 300 includes pressure sensors that are part of the seat 302 (e.g., the sensors 310 include pressure sensors), optical sensors that are part of the seat 302 or the back 304 (e.g., the sensors 310 and/or the sensors 312 include optical sensors), and/or electrical sensors that are part of the seat 302 or the back 304 (e.g., the sensors 310 and/or the sensors 312 include electrical sensors). In accordance with this embodiment, the risk factor evaluation component 116 can be configured to determine heart rate or heart rate variability based on the signals from such sensors. Heart rate variability correlates with risk of heart attack, particularly for patients that already have had heart attacks. Moreover, the heart rate and heart rate variability can loosely correlate with other cognitive and emotional parameters, which may be useful for entertainment and non-cardiovascular health (e.g., psychological health).

### Exemplary Embodiment 5

The chair 300 includes pressure sensors that are part of the back 304 (e.g., the sensors 312 include pressure sensors). Pursuant to this embodiment, the risk factor evaluation component 116 can analyze the signals from the pressure sensors in the back 304 of the chair 300 to monitor respiration.

### Exemplary Embodiment 6

The chair 300 includes multiple electrical sensors spread throughout the back 304 and the seat 302 of the chair 300 (e.g., the sensors 310 and the sensors 312 include electrical sensors). The electrical sensors in such embodiment can be employed to output signals that can be evaluated by the risk factor evaluation component 116 to determine thoracic impedance (e.g., the risk factor data 118). Thoracic impedance can be used to infer various cardiovascular properties, such as cardiac output, systemic vascular resistance and aortic blood flow velocity.

Accordingly, the multiple electrical sensors spread throughout the back 304 and the seat 302 of the chair 300 can be utilized to output signals indicative of electrical resistance across different lines through the torso and/or lower limbs of the user sitting on the chair 300. For instance, the risk factor evaluation component 116 can analyze differences in the electrical resistance between the electrical sensors over time. The changes in electrical resistance over time can be analyzed by the risk factor evaluation component 116 to measure various fluid phenomena pertaining to cardiovascular risk estimation.

### Exemplary Embodiment 7

The chair 300 includes acoustic sensors (e.g., the sensors 310, the sensors 312, and/or the sensors 314 can include acoustic sensors). By way of illustration, the acoustic sensors may be microphones, which can be placed in the back 304 or the seat 302 of the chair 300. The acoustic sensors may be suitable in place of pressure sensors or optical sensors for inferring pulse arrival time at different sites, for example. Moreover, the acoustic sensors may allow phonocardiography for assessing other cardiac risk indicators.

### Exemplary Embodiment 8

The chair 300 includes Doppler ultrasound sensors (e.g., the sensors 310, the sensors 312, and/or the sensors 314 include Doppler ultrasound sensors). The Doppler ultrasound sensors may allow direct determination of pulse wave velocity from the chair 300 from a single site. For instance, the Doppler ultrasound sensors can directly measure pulse wave velocity from an aorta of a user sitting in the chair 300. Use of ultrasound to measure pulse wave velocity can thus be incorporated into the chair 300.

With reference to FIGS. 5-6, illustrated is another exemplary chair 500 (e.g., the chair 102 of FIGS. 1-2) that senses cardiovascular risk factors. As depicted, the chair 500 is a recliner. FIG. 5 shows a side view of the chair 500 in an upright position, and FIG. 6 shows a side view of the chair 500 in a reclined position.

Similar to the chair 300 of FIGS. 3-4, the chair 500 includes a seat 502, a back 504, arms 506, and a support 508. Moreover, similar to the chair 300 of FIGS. 3-4, the chair 500 optionally includes one or more sensors 510 that are part of the seat 502, optionally includes one or more sensors 512 that are part of the back 504 and/or optionally includes one or more sensors 514 that are part of the arms 506.

The chair 500 further includes a leg rest 516 and a lever 518. The lever 518 can be manipulated to cause the chair 500 to transition from the upright position as shown in FIG. 5 to the reclined position as shown in FIG. 6 (and vice versa). When in the upright position, the back 504 is upright and the leg rest 516 is retracted inwards. Further, when in the reclined position, the back 504 is reclined and the leg rest 516 is extended outwards.

The signals output by the sensors of the chair 500 (e.g., the sensor(s) 510, the sensor(s) 512, and/or the sensor(s) 514) can be analyzed by the risk factor evaluation component 116 to detect an ability of a user to compensate for changes in posture. The signals output by the sensors of the chair 500 can be analyzed by the risk factor evaluation component 116 to measure heart rate, blood pressure, etc. when sitting (e.g., with the chair 500 in the upright position as shown in FIG. 5) and when reclined (e.g., with the chair 500 in the reclined position as shown in FIG. 6). Moreover, the risk factor evaluation component 116 can compare the measurements for the user when sitting versus when reclined to evaluate the ability of the user to compensate for changes in posture. Thus, the risk factor evaluation component 116 can analyze baroreflex sensitivity (BRS) (e.g., the risk factor data 118) of the user based on changes in the measurements before and after reclining the chair 500.

According to various examples, it is contemplated that the chair 500 can be manipulated manually by a user utilizing the lever 518. Following this example, the position detection component 114 can identify the transition between the positions and trigger measurement of the heart rate, blood pressure, etc. However it is to be appreciated that in accordance with other examples, the chair can be automatically transitioned between the upright and reclined positions (e.g., periodically to test BRS, etc.).

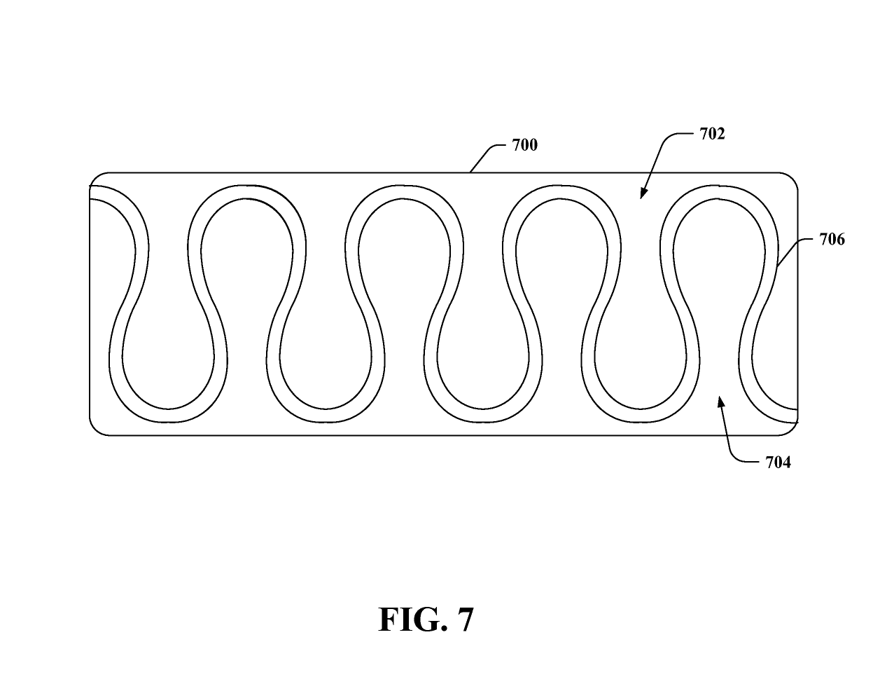

Turning to FIG. 7, illustrated is a top view of an exemplary arm 700 of a chair (e.g., the chair 102 of FIGS. 1-2, the chair 300 of FIGS. 3-4, the chair 500 of FIGS. 5-6, etc.). The arm 700 includes an electrical sensor. More particularly, the arm 700 includes a first electrode 702 and a second electrode 704 that are non-touching. The first electrode 702 and the second electrode 704 are separated by a spacer 706.

The first electrode 702 is formed of a first electrically conductive material and the second electrode 704 is formed of a second electrically conductive material. According to an example, the first electrically conductive material and the second electrically conductive material can be the same (e.g., the first electrode 702 and the second electrode 704 can be formed of the same electrically conductive material). Pursuant to another example, the first electrically conductive material can differ from the second electrically conductive material (e.g., the first electrode 702 and the second electrode 704 can be formed of differing electrically conductive materials). Moreover, the spacer 706 is formed of a non-conductive material.

The first electrode 702 and the second electrode 704 are arranged in an interlocking zigzag pattern. A width of the interlocking zigzag pattern of the first electrode 702 and the second electrode 704 can enable an arm (e.g., elbow) of a user sitting in the chair to touch both the first electrode 702 and the second electrode 704 regardless of location of the arm of the user (e.g., a circle having a 35 mm diameter positioned on the arm 700 touches both the first electrode 702 and the second electrode 704 regardless of location with respect to the arm 700). For instance, the electrode pattern for the arm 700 of the chair can be employed to obtain an EKG signal when the user is sitting in the chair.

According to an example, the electrode being used for the driven right leg (DRL) can be on an outside of the arm 700 (e.g., on the top of the arm away from a side at which the user sits) to increase a chance that the user sitting in the chair is in contact with both of the opposing leads. Moreover, contact with only one of the active ground fabrics may be needed.

An upper surface (the surface shown in FIG. 7) of the arm 700 can be an EKG contact (e.g., the upper surface of the arm 700 can be a cover of an armrest). Moreover, the first electrode 702 and the second electrode 704 can be formed of differing types of fabrics. According to an example, the first electrode 702 (e.g., active ground) can be formed of a first electrically conductive material such as copper taffeta of 0.05 Ohm/sq, and the second electrode 704 (e.g., opposing lead) can be formed of a second electrically conductive material such as silver ribstop of 0.25 Ohm/sq. In accordance with another example, the first electrode 702 and the second electrode 704 can be made of the same fabric, as long as the two pieces of fabric on the arm 700 do not touch (e.g., separated by the spacer 706). Moreover, other electrically conductive materials that can be used for one or both of the electrodes 702 and 704, such as copper tape.

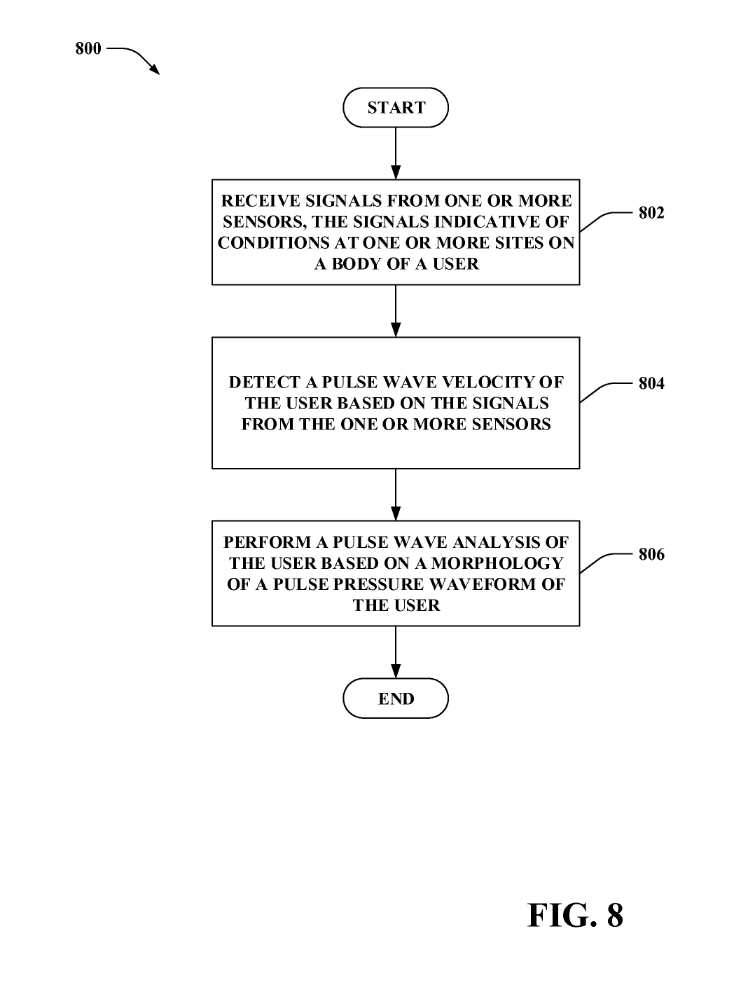

FIG. 8 illustrates an exemplary methodology relating to sensing cardiovascular risk factors. While the methodology is shown and described as being a series of acts that are performed in a sequence, it is to be understood and appreciated that the methodology is not limited by the order of the sequence. For example, some acts can occur in a different order than what is described herein. In addition, an act can occur concurrently with another act. Further, in some instances, not all acts may be required to implement a methodology described herein.

Moreover, the acts described herein may be computer-executable instructions that can be implemented by one or more processors and/or stored on a computer-readable medium or media. The computer-executable instructions can include a routine, a sub-routine, programs, a thread of execution, and/or the like. Still further, results of acts of the methodologies can be stored in a computer-readable medium, displayed on a display device, and/or the like.

FIG. 8 illustrates a methodology 800 for sensing cardiovascular risk factors. At 802, signals from one or more sensors can be received. A chair can include the one or more sensors; however, it is also contemplated that a peripheral device communicatively connected to the chair can include at least one of the one or more sensors from which the signals are received. Further, the signals can be indicative of conditions at one or more sites on a body of a user. At 804, a pulse wave velocity of the user can be detected based on the signals from the one or more sensors. At 806, a pulse wave analysis of the user can be performed based on a morphology of a pulse pressure waveform of the user. Further, the pulse pressure waveform can be detected based on the signals from the one or more sensors.

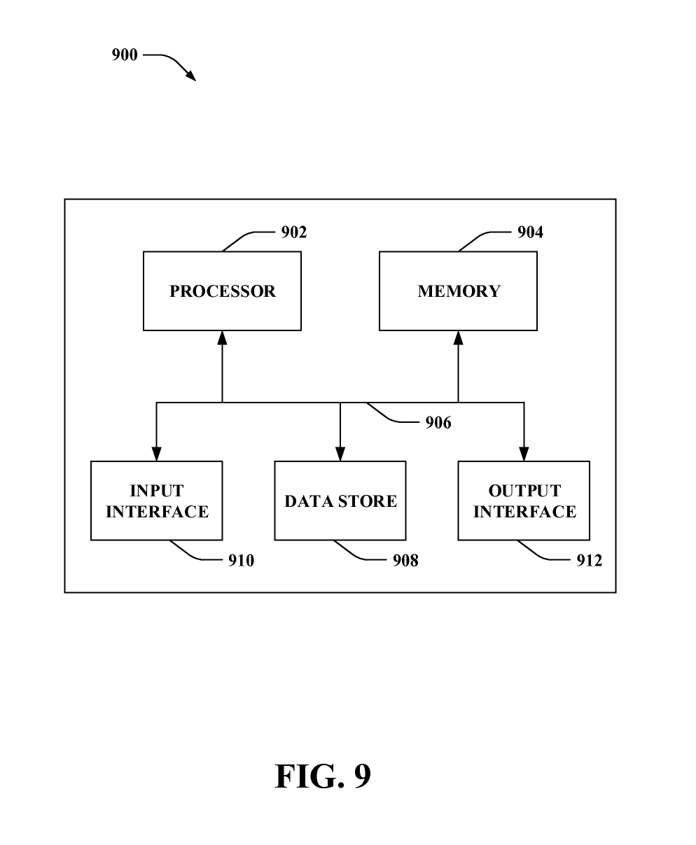

Referring now to FIG. 9, a high-level illustration of an exemplary computing device 900 that can be used in accordance with the systems and methodologies disclosed herein is illustrated. For instance, the computing device 900 may be used in a system that senses cardiovascular risk factors. By way of example, a chair (e.g., the chair 102 of FIGS. 1-2, the chair 300 of FIGS. 3-4, the chair 500 of FIGS. 5-6, etc.) can include the computing device 900. Pursuant to another example, the computing device 900 can be or include the computing system 124. The computing device 900 includes at least one processor 902 that executes instructions that are stored in a memory 904. The instructions may be, for instance, instructions for implementing functionality described as being carried out by one or more components discussed above or instructions for implementing one or more of the methods described above. The processor 902 may access the memory 904 by way of a system bus 906. In addition to storing executable instructions, the memory 904 may also store risk factor data, signals output by sensor(s) of a chair, signals output by external sensor(s), and so forth.

The computing device 900 additionally includes a data store 908 that is accessible by the processor 902 by way of the system bus 906. The data store 908 may include executable instructions, risk factor data, signals output by sensor(s) of a chair, signals output by external sensor(s), etc. The computing device 900 also includes an input interface 910 that allows external devices to communicate with the computing device 900. For instance, the input interface 910 may be used to receive instructions from an external computer device, from a user, etc. The computing device 900 also includes an output interface 912 that interfaces the computing device 900 with one or more external devices. For example, the computing device 900 may display text, images, etc. by way of the output interface 912.

It is contemplated that the external devices that communicate with the computing device 900 via the input interface 910 and the output interface 912 can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 900 in a manner free from constraints imposed by input device such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 900 may be a distributed system. Thus, for instance, several devices may be in communication by way of a network connection and may collectively perform tasks described as being performed by the computing device 900.

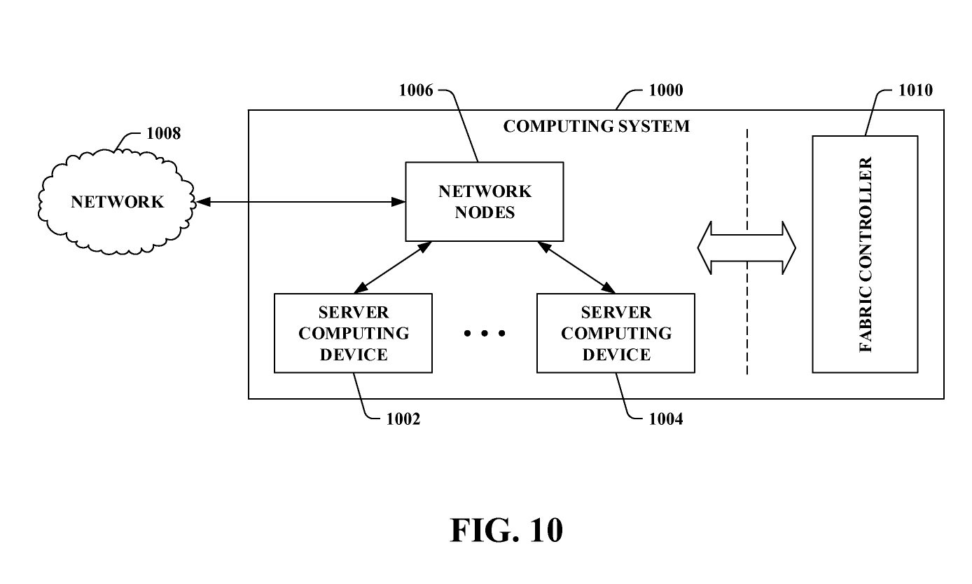

Turning to FIG. 10, a high-level illustration of an exemplary computing system 1000 that can be used in accordance with the systems and methodologies disclosed herein is illustrated. For instance, the computing system 1000 can be or include the computing system 124. Additionally or alternatively, the computing system 124 can be or include the computing system 1000.

The computing system 1000 includes a plurality of server computing devices, namely, a server computing device 1002, . . . , and a server computing device 1004 (collectively referred to as server computing devices 1002-1004). The server computing device 1002 includes at least one processor and a memory; the at least one processor executes instructions that are stored in the memory. The instructions may be, for instance, instructions for implementing functionality described as being carried out by one or more components discussed above or instructions for implementing one or more of the methods described above. Similar to the server computing device 1002, at least a subset of the server computing devices 1002-1004 other than the server computing device 1002 each respectively include at least one processor and a memory. Moreover, at least a subset of the server computing devices 1002-1004 include respective data stores.

Processor(s) of one or more of the server computing devices 1002-1004 can be or include the processor 202. Further, a memory (or memories) of one or more of the server computing devices 1002-1004 can be or include the memory 204. Moreover, a data store (or data stores) of one or more of the server computing devices 1002-1004 can be or include the data store 206.

The computing system 1000 further includes various network nodes 1006 that transport data between the server computing devices 1002-1004. Moreover, the network nodes 1002 transport data from the server computing devices 1002-1004 to external nodes (e.g., external to the computing system 1000) by way of a network 1008. The network nodes 1002 also transport data to the server computing devices 1002-1004 from the external nodes by way of the network 1008. The network 1008, for example, can be the Internet, a cellular network, or the like. The network nodes 1006 include switches, routers, load balancers, and so forth.

A fabric controller 1010 of the computing system 1000 manages hardware resources of the server computing devices 1002-1004 (e.g., processors, memories, data stores, etc. of the server computing devices 1002-1004). The fabric controller 1010 further manages the network nodes 1006. Moreover, the fabric controller 1010 manages creation, provisioning, de-provisioning, and supervising of virtual machines instantiated upon the server computing devices 1002-1004.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something.”

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and blu-ray disc (BD), where disks usually reproduce data magnetically and discs usually reproduce data optically with lasers. Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

What is claimed is:

1. A system that senses cardiovascular risk factors of a user, comprising:
a chair, comprising:
one or more sensors configured to output signals indicative of conditions at one or more sites on a body of the user, wherein at least one of a seat of the chair, a back of the chair, or arms of the chair comprises the one or more sensors; and
a collection circuit configured to receive the signals from the one or more sensors; and
a risk factor evaluation component configured to:
detect a pulse wave velocity of the user based on the signals from the one or more sensors; and
perform a pulse wave analysis of the user based on a morphology of a pulse pressure waveform of the user, the pulse pressure waveform detected based on the signals from the one or more sensors.

2. The system of claim 1, the risk factor evaluation component configured to derive at least one of a pulse pressure, a blood pressure, an augmentation index, or a systolic ejection time of the user via the pulse wave analysis.

3. The system of claim 1, the risk factor evaluation component configured to infer thoracic impedance based on the signals from the one or more sensors.

4. The system of claim 1, wherein:
the one or more sensors comprise one or more pressure sensors that are part of the seat; and
the risk factor evaluation component configured to detect the pulse pressure waveform of the user based on signals from the one or more pressure sensors that are part of the seat.

5. The system of claim 1, wherein:
the one or more sensors comprise optical sensors that are part of the back; and
the risk factor evaluation component configured to detect the pulse wave velocity of the user based on signals from the optical sensors that are part of the back.

6. The system of claim 5, wherein:
the one or more sensors further comprise at least one of optical sensors that are part of the seat or pressure sensors that are part of the seat; and
the risk factor evaluation component configured to detect the pulse wave velocity of the user further based on signals from the optical sensors that are part of the seat or signals from the pressure sensors that are part of the seat.

7. The system of claim 1, wherein:
the one or more sensors comprise:
electrical sensors that are part of the arms; and
at least one of pressure sensors that are part of the seat, optical sensors that are part of the seat, or optical sensors that are part of the back; and
the risk factor evaluation component configured to detect the pulse wave velocity of the user based on:
signals from the electrical sensors that are part of the arms; and
signals from the pressure sensors that are part of the seat, signals from the optical sensors that are part of the seat, or signals from the optical sensors that are part of the back.

8. The system of claim 7, the risk factor evaluation component configured to:
measure an electrocardiogram (EKG) signal of the user based on the signals from the electrical sensors that are part of the arms;
detect a first time at which a pulse leaves a heart of the user based on the EKG signal;
infer a second time at which the pulse arrives at a different site on the body of the user based on the signals from the pressure sensors that are part of the seat, the signals from the optical sensors that are part of the seat, or the signals from the optical sensors that are part of the back;
compute a pulse transit time as a difference between the first time and the second time; and
compute the pulse wave velocity based on the pulse transit time and a distance between the heart and the different site on the body of the user.

9. The system of claim 1, wherein:
the one or more sensors comprise at least one of pressure sensors that are part of the seat, optical sensors that are part of the seat, or optical sensors that are part of the back;
the collection circuit further configured to receive signals from electrical sensors that are part of a peripheral device communicatively connected to the chair; and
the risk factor evaluation component configured to detect the pulse wave velocity of the user based on:
signals from the electrical sensors that are part of the peripheral device; and
signals from the pressure sensors that are part of the seat, signals from the optical sensors that are part of the seat, or signals from the optical sensors that are part of the back.

10. The system of claim 1, wherein the one or more sensors comprise electrical sensors that are part of the arms, each of the arms comprising:
a first electrode formed of a first electrically conductive material;
a second electrode formed of a second electrically conductive material;
a spacer formed of a non-conductive material;
the first electrode and the second electrode being separated by the spacer; and
the first electrode and the second electrode arranged in an interlocking zigzag pattern.

11. The system of claim 1, the chair comprising:
at least one processor; and
memory comprising the risk factor evaluation component, the risk factor evaluation component being executable by the processor.

12. The system of claim 1, further comprising:
a computing system, comprising:
at least one processor; and
memory that comprises the risk factor evaluation component, the risk factor evaluation component being executable by the processor; and
the chair further comprising:
a communication circuit that sends the signals from the one or more sensors to the computing system.

13. A chair, comprising:
a seat;
one or more pressure sensors configured to output signals indicative of conditions at one or more sites on a body of a user, the seat comprising the one or more pressure sensors;
a collection circuit configured to receive the signals from the one or more pressure sensors; and
a risk factor evaluation component configured to:
detect a pulse pressure waveform of the user based on the signals from the one or more pressure sensors; and
perform a pulse wave analysis of the user based on a morphology of the pulse pressure waveform of the user.

14. The chair of claim 13, the risk factor evaluation component configured to derive at least one of a pulse pressure, a blood pressure, an augmentation index, or a systolic ejection time of the user via the pulse wave analysis.

15. The chair of claim 13, wherein the morphology comprises:
shape features of the pulse pressure waveform, the shape features comprise a derivative and a peak-to-peak amplitude; and
waveform landmarks of the pulse pressure waveform, the waveform landmarks comprise a systolic peak, a diastolic foot, a dicrotic notch, and an anacrotic notch.

16. The chair of claim 13, further comprising:
at least one of electrical sensors that are part of arms of the chair or the collection circuit configured to receive signals from electrical sensors that are part of a peripheral device communicatively connected to the chair;
the risk factor evaluation component configured to detect a pulse wave velocity of the user based on:
signals from the electrical sensors that are part of the arms or the signals from the electrical sensors that are part of the peripheral device; and
the signals from the one or more pressure sensors.

17. A chair, comprising:
at least one of electrical sensors that are part of arms of the chair or a collection circuit configured to receive signals from electrical sensors that are part of a peripheral device communicatively connected to the chair;
at least one of pressure sensors that are part of a seat of the chair, optical sensors that are part of the seat, or optical sensors that are part of a back of the chair; and
a risk factor evaluation component configured to detect a pulse wave velocity of a user based on:
signals from the electrical sensors that are part of the arms or the signals from the electrical sensors that are part of the peripheral device; and
signals from the pressure sensors that are part of the seat, signals from the optical sensors that are part of the seat, or signals from the optical sensors that are part of the back.

18. The chair of claim 17, the risk factor evaluation component configured to:
measure an electrocardiogram (EKG) signal of the user based on the signals from the electrical sensors that are part of the arms or the electrical sensors that are part of the peripheral device;
detect a first time at which a pulse leaves a heart of the user based on the EKG signal;
infer a second time at which the pulse arrives at a different site on the body of the user based on the signals from the pressure sensors that are part of the seat, the optical sensors that are part of the seat, or the optical sensors that are part of the back;
compute a pulse transit time as a difference between the first time and the second time; and
compute the pulse wave velocity based on the pulse transit time and a distance between the heart and the different site on the body of the user.

19. The chair of claim 17, further comprising the electrical sensors that are part of the arms, each of the arms comprising:
a first electrode formed of a first electrically conductive material;
a second electrode formed of a second electrically conductive material;
a spacer formed of a non-conductive material;
the first electrode and the second electrode being separated by the spacer; and
the first electrode and the second electrode arranged in an interlocking zigzag pattern.

20. The chair of claim 17, further comprising the pressure sensors that are part of the seat of the chair;
the risk factor evaluation component configured to:
detect a pulse pressure waveform of the user based on the signals from the pressure sensors; and
perform a pulse wave analysis of the user based on a morphology of the pulse pressure waveform of the user.
